SEQUENCE LISTING (12 sequences)

SEQ ID NO: 1 - DNA, 67 bp, artificial
  Reverse Transcription Oligo SCT01
ctgatcgtcggactgtagaactctcaagcagaagacggcatacgattttttttttttttttttttvn

SEQ ID NO: 2 - DNA, 67 bp, artificial
  Reverse Transcription Oligo MCA02
cagatcgtcggactgtagaactctcaagcagaagacggcatacgattttttttttttttttttttvn

SEQ ID NO: 3 - DNA, 67 bp, artificial
  Reverse Transcription Oligo LGT03
gtgatcgtcggactgtagaactctcaagcagaagacggcatacgattttttttttttttttttttvn

SEQ ID NO: 4 - DNA, 67 bp, artificial
  Reverse Transcription Oligo HTC04
tcgatcgtcggactgtagaactctcaagcagaagacggcatacgattttttttttttttttttttvn

SEQ ID NO: 5 - DNA, 67 bp, artificial
  Reverse Transcription Oligo YAG05
aggatcgtcggactgtagaactctcaagcagaagacggcatacgattttttttttttttttttttvn

SEQ ID NO: 6 - DNA, 18 bp, artificial
  Primer qNTI200
caagcagaagacggcata

SEQ ID NO: 7 - DNA, 46 bp, artificial
  Primer qNTI201
aatgatacggcgaccaccgacaggttcagagttctacagtccgacg

SEQ ID NO: 8 - DNA, 32 bp, artificial
  sequencing primer
cgacaggttcagagttctacagtccgacgatc

SEQ ID NO: 9 - DNA, 37 bp, artificial
  Oligo ccdc124F
ggcgccaagcttggaggcgcgaccgggccggcgctgg

SEQ ID NO: 10 - DNA, 37 bp, artificial
  Oligo ccdc124R
ggcgccctcgagttgggggcattgaagggcacggccc

SEQ ID NO: 11 - DNA, 37 bp, artificial
  oligo rnd3F
ggcgccaagcttcagtcggctcggaattggacttggg

SEQ ID NO: 12 - DNA, 37 bp, artificial
  oligo rnd3R
ggcgccctcgagctattctgcaccctggaggcgtagc

(19) United States
(12) Patent Application Publication
(10) Pub. No.: US 2013/0096012 A1
(43) Pub. Date: Apr. 18, 2013

(54) METHOD AND KIT FOR IDENTIFYING A TRANSLATION INITIATION SITE ON AN MRNA

(71) Applicant: Cornell University, Ithaca, NY (US)

(72) Inventors: Shu-Bing Qian, Ithaca, NY (US); Sooncheol Lee, Ithaca, NY (US); Botao Liu, Ithaca, NY (US)

(73) Assignee: CORNELL UNIVERSITY, Ithaca, NY (US)

(21) Appl. No.: 13/625,495

(22) Filed: Sep. 24, 2012

Related U.S. Application Data
(60) Provisional application No. 61/538,848, filed on Sep. 24, 2011.

(51) Int. Cl. C12Q 1/68

(52) U.S. Cl. CPC C12Q 1/68
USPC 506/2; 435/6.1; 435/6.12

(57) ABSTRACT

The present invention relates to a method and kit for identifying a translation initiation site on an mRNA. The method involves contacting a first mRNA with a first translation inhibitor to preferentially stabilize one or more initiation ribosomes at translation initiation sites on the first mRNA. A second mRNA is contacted with a second translation inhibitor different from the first translation inhibitor to stabilize one or more initiation ribosomes and one or more elongation ribosomes on the second mRNA. The location of ribosomes stabilized on the first mRNA is compared to the location of ribosomes stabilized on the second mRNA.

This application claims the benefit of U.S. Provisional Patent Application Ser. No. 61/538,848, filed Sep. 24, 2011, which is hereby incorporated by reference in its entirety.

This invention was made with government support under NIH grant numbers CA106150 and 1 DP2 OD006449-01 and DOD grant number TS10078. The government has certain rights in the invention.

### FIELD OF THE INVENTION

The present invention relates to a method and kit for identifying a translation initiation site on an mRNA.

### BACKGROUND OF THE INVENTION

Protein synthesis is the final step in the flow of genetic information and lies at the heart of cellular metabolism. Translation is principally regulated at the initiation stage and there has been significant progress over the last decade in dissecting the role of initiation factors (“eIFs”) in the assembly of elongation-competent 80S ribosomes (Sonenberg et al., “Regulation of Translation Initiation in Eukaryotes: Mechanisms and Biological Targets,” Cell 136(4):731-745 (2009); Jackson et al., “The Mechanism of Eukaryotic Translation Initiation and Principles of its Regulation,” Nat. Rev. Mol. Cell Biol. 11(2):113-127 (2010); and Gray et al., “Control of Translation Initiation in Animals,” Annu. Rev. Cell Dev. Biol. 14:399-458 (1998)). However, mechanisms underlying start codon recognition are not fully understood. Proper selection of the translation initiation site (“TIS”) on mRNAs is crucial for the production of desired protein products. A fundamental and long-sought goal in understanding translational regulation is the precise determination of TIS codons across the entire transcriptome.

In eukaryotes, ribosomal scanning is a well-accepted model for start codon selection (Kozak “Pushing the Limits of the Scanning Mechanism for Initiation of Translation,” Gene 299(1-2):1-34 (2002)). During cap-dependent translation initiation, the small ribosome subunit (40S) is recruited to the 5′ end of mRNA (the m⁷G cap) in the form of a 43S pre-initiation complex (“PIC”). The PIC is thought to scan along the message in search for the start codon. It is commonly assumed that the first AUG codon that the scanning PIC encounters serves as the start site for translation. However, many factors influence the start codon selection. For instance, the initiator AUG triplet is usually in an optimal context with a purine at position −3 and a guanine at position +4 (Kozak, “Structural Features in Eukaryotic mRNAs that Modulate the Initiation of Translation,” J. Biol. Chem. 266(30):19867-19870 (1991)). The presence of mRNA secondary structure at or near the TIS position also influences the recognition efficiency (Kozak, “Downstream Secondary Structure Facilitates Recognition of Initiator Codons by Eukaryotic Ribosomes,” Proc. Natl. Acad. Sci. U.S.A. 87(21):8301-8305 (1990)). In addition to these cis sequence elements, the stringency of TIS selection is also subject to regulation by trans acting factors such as eIF1 and eIF1A (Maag et al., “A Conformational Change in the Eukaryotic Translation Preinitiation Complex and Release of eIF1 Signal Recognition of the Start Codon,” Mol. Cell 17(2):265-275 (2005); and Martin-Marcos et al., “Functional Elements in Initiation Factors 1, 1A, and 2beta Discriminate Against Poor AUG Context and Non-AUG Start Codons,” Mol. Cell Biol. 31(23):4814-4831 (2011)). Inefficient recognition of an initiator codon results in a portion of 43S PIC continuing to scan and initiating at a downstream site, in a process known as leaky scanning (Kozak “Pushing the Limits of the Scanning Mechanism for Initiation of Translation,” Gene 299(1-2):1-34 (2002)). However, little is known about the frequency of leaky scanning events at the transcriptome level.

Many recent studies have uncovered a surprising variety of potential translation start sites upstream of the annotated coding sequence (“CDS”) (Iacono et al., “uAUG and uORFs in Human and Rodent 5′ Untranslated mRNAs,” Gene 349:97-105 (2005) and Morris et al., “Upstream Open Reading Frames as Regulators of mRNA Translation,” Mol. Cell Biol. 20(23):8635-8642 (2000)). It has been estimated that about 50% of mammalian transcripts contain at least one upstream open reading frame (“uORF”) (Calvo et al., “Upstream Open Reading Frames Cause Widespread Reduction of Protein Expression and are Polymorphic Among Humans,” Proc. Natl. Acad. Sci. U.S.A. 106(18):7507-7512 (2009) and Resch et al., “Evolution of Alternative and Constitutive Regions of Mammalian 5′ UTRs,” BMC Genomics 10:162 (2009)). Intriguingly, many non-AUG triplets have been reported to act as alternative start codons for initiating uORF translation (Touriol et al., “Generation of Protein Isoform Diversity by Alternative Initiation of Translation at Non-AUG Codons,” Biol. Cell 95(3-4):169-178 (2003)). Since there is no reliable way to predict non-AUG codons as potential initiators from in silico sequence analysis, there is an urgent need to develop experimental approaches for genome-wide TIS identification.

Ribosome profiling, based on deep sequencing of ribosome-protected mRNA fragments (“RPF”), has proven to be powerful in defining ribosome positions on the entire transcriptome (Ingolia et al., “Genome-Wide Analysis in vivo of Translation with Nucleotide Resolution using Ribosome Profiling,” Science 324(5924):218-223 (2009) and Guo et al., “Mammalian MicroRNAs Predominantly Act to Decrease Target mRNA Levels,” Nature 466(7308):835-840 (2010)). However, the standard ribosome profiling is not suitable for TIS identification. Elevated ribosome density near the beginning of CDS does not allow for unambiguous identification of alternative TIS positions, in particular the TIS positions associated with overlapping ORFs. To overcome this problem, a recent study used an initiation-specific translation inhibitor harringtonine to deplete elongating ribosomes from mRNAs (Ingolia et al., “Ribosome Profiling of Mouse Embryonic Stem Cells Reveals the Complexity and Dynamics of Mammalian Proteomes,” Cell 147(4):789-802 (2011)). This approach uncovered an unexpected abundance of alternative TIS codons, in particular non-AUG codons, in the 5′UTR. However, since the inhibitory mechanism of harringtonine on the initiating ribosome is unclear, it remains to be confirmed whether the harringtonine-marked TIS codons truly represent physiological translation initiation sites.

The present invention is directed to overcoming deficiencies in the art.

### SUMMARY OF THE INVENTION

One aspect of the present invention relates to a method for identifying a translation initiation site on an mRNA. This method involves providing a first mRNA in an environment suitable for translation. The first mRNA is contacted with a first translation inhibitor to preferentially stabilize one or more initiation ribosomes at translation initiation sites on the first mRNA. A second mRNA is provided in an environment suitable for translation, where the second mRNA has a nucleotide sequence that is substantially similar to a nucleotide sequence of the first mRNA. The second mRNA is contacted with a second translation inhibitor different from the first translation inhibitor to stabilize one or more initiation ribosomes and one or more elongation ribosomes on the second mRNA. The location of ribosomes stabilized on the first mRNA is compared to the location of ribosomes stabilized on the second mRNA, where ribosomes stabilized at a location on the first mRNA at a higher density than ribosomes stabilized at the same location on the second mRNA identifies the location as a translation initiation site on the first and second mRNAs.

Another aspect of the present invention relates to a kit for identifying a translation initiation site on an mRNA. The kit includes a first translation inhibitor capable of preferentially stabilizing initiation ribosomes at translation initiation sites on an mRNA. Also included in the kit is a second translation inhibitor different from the first translation inhibitor, where the second translation inhibitor is capable of stabilizing initiation ribosomes and elongation ribosomes on an mRNA. The kit also includes instructions for (i) contacting a first mRNA with the first translation inhibitor and a second mRNA with the second translation inhibitor and (ii) comparing the location of ribosomes stabilized on the first mRNA to ribosomes stabilized on the second mRNA to identify translation initiation sites on the first and second mRNAs.

The present invention relates to a global translation initiation sequencing (“GTI-seq”) by utilizing (at least) two related but distinct translation inhibitors to effectively differentiate ribosome initiation from elongation. GTI-seq has the potential to reveal a comprehensive and unambiguous set of TIS codons at near single nucleotide resolution. The resulting TIS maps provide a remarkable display of alternative translation initiators that vividly delineates the variation in start codon selection. This allows for a more complete assessment of the underlying principles that specify start codon usage in vivo.

### BRIEF DESCRIPTION OF THE DRAWINGS

FIGS. 1A-D show the experimental strategy of GTI-seq using ribosome E-site translation inhibitors. Specifically, FIG. 1A is a schematic diagram of the experimental design for GTI-seq. Translation inhibitors cycloheximide (“CHX”) and lactimidomycin (“LTM”) bind to the ribosome E-site, resulting in inhibition of translocation. While CHX binds to all translating ribosomes (FIG. 1A, left panel), LTM preferably incorporates into the initiating ribosomes when the E-site is free of tRNA (FIG. 1A, right panel). FIG. 1B is a schematic illustration showing ribosome profiling using CHX and LTM side-by-side, which allows distinguishing the initiating ribosome from the elongating one. FIG. 1C is a graph showing data related to HEK293 cells being treated with either DMSO, 100 μM CHX, or 50 μM LTM for 30 min before ribosome profiling. Normalized RPF reads were averaged across the entire transcriptome, aligned at either their start site or stop codon. FIG. 1D presents two graphs showing metagene analysis of RPFs obtained from HEK293 cells treated with either harringtonine (FIG. 1D, left panel) or LTM (FIG. 1D, right panel). All mapped reads were aligned at the annotated start codon AUG, and the reads density at each nucleotide position was averaged using the P-site of RPFs.

FIGS. 2A-D show global identification of TIS by GTI-seq. In particular, FIG. 2A sets forth graphs to show TIS identification on the PYCR1 transcript. Both LTM and CHX reads were plotted as grey bar graphs. TIS identification was based on normalized LTM reads density subtracted by CHX reads density. All three reading frames were separated and presented. Identified TIS position is marked by an asterisk. The annotated coding region is illustrated by start codon (grey triangle) and stop codon (black triangle). FIG. 2B presents two pie graphs showing codon composition of all TIS codons identified by GTI-seq (FIG. 2B, left panel) in comparison to the overall codon distribution over the entire transcriptome (FIG. 2B, right panel). FIG. 2C is a histogram showing the overall distribution of TIS number identified on each transcript. FIG. 2D presents graphs showing mis-annotation of the start codon on the CLK3 transcript. The annotated coding region is illustrated by start codon (grey triangle) and stop codon (black triangle). AUG codons on the body of the coding region are also shown as empty triangles. Only one reading frame is shown for clarity.

FIGS. 3A-F relate to the characterization of downstream TIS (“dTIS”). FIG. 3A provides graphs showing identification of multiple TIS codons on the AIMP1 transcript. Only one reading frame is shown for clarity. FIG. 3B is a pie graph showing codon composition of total dTIS codons identified by GTI-seq. FIG. 3C is a schematic illustration and graph showing relative initiation efficiency at the first AUG codon with different sequence context (one-tailed Wilcoxon Ranksum test, Strong vs. Weak: p=7.92×10⁻²⁴; Weak vs. No-Kozak: p=1.34×10⁻⁷⁵). FIG. 3D shows how genes are grouped according to the identified initiation at either aTIS, dTIS, or both. Sequence context surrounding the aTIS is shown as Sequence Logos. Chi-square test, p=2.57×10⁻¹⁰⁰ for −3 position and p=3.95×10⁻¹⁸ for +4 position. FIG. 3E shows the identification of multiple TIS codons on the CCDCl24 transcript. FIG. 3F provides evidence of the validation of CCDCl24TIS codons by immunoblotting. The DNA fragment encompassing both the 5′UTR and the CDS of CCDCl24 was cloned and transfected into HEK 293 cells. Whole cell lysates were immunoblotted using c-myc antibody.

FIGS. 4A-D relate to the characterization of upstream TIS (“uTIS”). FIG. 4A shows the identification of multiple TIS codons on the ATF4 transcript. Insert: a region of frame 0 with 10× enlarged Y-axis, showing the LTM peak at the annotated start codon AUG. Different ORFs are shown in boxes. FIG. 4B is a pie graph showing codon composition of total uTIS codons identified by GTI-seq. FIG. 4C shows the identification of multiple TIS codons on the RND3 transcript. FIG. 4D shows the validation of RND3TIS codons by immunoblotting. The DNA fragment encompassing both the 5′UTR and the CDS of RND3 was cloned and transfected into HEK 293 cells. Whole cell lysates were immunoblotted using c-myc antibody.

FIGS. 5A-C show the impact of uORF features on translational regulation. In particular, FIG. 5A shows the sequence composition of uTIS codons for genes with or without aTIS initiation. Genes are classified into two groups based on aTIS initiation, and the uTIS sequence composition is categorized based on the consensus features shown on the right. The graphs of FIG. 5B show the contribution of mRNA secondary structure to TIS selection. Genes are grouped based on uTIS codon features listed in FIG. 5A. For each group, the transcripts with (grey line) or without (black line) aTIS initiation are analyzed for the averaged ΔG value in regions surrounding the identified uTIS codons. FIG. 5C shows the composition of uORFs in gene groups with or without aTIS initiation on their transcripts. Different ORF features are shown on the right.

FIGS. 6A-G relate to cross-species conservation of alternative TIS positions and identification of translated non-coding RNA (“ncRNA”). FIG. 6A shows the evolutionary conservation of alternative TIS positions identified by GTI-seq in HEK293 and MEF cells. Alternative uTIS and dTIS positions identified on human-mouse ortholog mRNA pairs are each classified into two subsets according to the alignment score of relevant sequences (5′UTR for uTIS and CDS for dTIS). Each subset is further divided based on types of alternative ORFs. Percentage values are presented in the table of FIG. 6A. FIG. 6B shows the conservation of uTIS positions on the RNF10 transcript with high sequence similarity of 5′UTR between HEK293 and MEF cells. The dark grey region indicates matched sequences, black is used for mismatched ones, and light grey for sequence gaps. Identified uTIS positions are indicated by triangles. FIG. 6C shows the conservation of uTIS positions on the CTTN transcript with low sequence similarity of 5′UTR between HEK293 and MEF cells. FIG. 6D is a pie chart showing the relative percentage of mRNA, ncRNA, and translated ncRNA identified by GTI-seq. FIG. 6E is a histogram showing the overall length distribution of ORF identified in ncRNAs. FIG. 6F shows the identification of multiple TIS positions on the ncRNA LOC100499177. FIG. 6G is a graph showing the evolutionary conservation of ORF region on ncRNAs identified by GTI-seq. PhastCons scores are retrieved from the primate genome sequence alignment.

FIG. 7 is a series of graphs showing polysome profile analysis in cells treated with ribosome E-site translation inhibitors. In particular, HEK293 cells were pre-treated with equal volume of DMSO, 100 μM CHX, or 50 μM LTM for 30 min followed by sucrose gradient sedimentation. Both 80S monosome and polysome peaks are indicated.

FIGS. 8A-C are a series of graphs showing metagene analysis of RPFs obtained using different approaches. RPF reads previously reported using harringtonine in mouse embryonic stem cells were replotted after peptidyl (P)-site adjustment based on the original report (HRT1, FIG. 8A). RPF reads obtained from HEK293 cells treated with either harringtonine (HRT2, FIG. 8B) or LTM (FIG. 8C) were plotted by applying a 12-nt offset to reads with a length range of 26-29 nt. All mapped reads are aligned at the annotated start codon AUG, and the reads density at each nucleotide position is averaged using the P-site of RPFs.

FIG. 9 is a graph showing false-positive and false-negative rates at various RLTM-RCHX thresholds. The false-negative rate is computed as the percentage of undetected aTIS among the top 10% translated aTIS codons based on CHX reads within five codons downstream of the aTIS. The lower and upper bounds of false-negative rate are determined by either including or excluding the cases having a dTIS within five codons and/or a uORF overlapping aTIS. The false-positive rate is computed as the percentage detected among strictly untranslated aTIS codons with either no CHX reads (CHX=0) or fewer than five CHX reads (CHX <5) within five codons downstream of the aTIS.

FIGS. 10A-C relate to global TIS identification in MEF cells. FIG. 10A is a graph showing codon composition of all TIS codons identified by GTI-seq in MEF cells. FIG. 10B is a graph showing codon composition of uTIS codons identified by GTI-seq in MEF cells. FIG. 10C is a histogram showing the overall distribution of the number of TIS positions identified on each transcript from MEF cells.

FIG. 11 is a table showing conservation of alternative TIS positions between human and mouse cells. Alternative TIS positions identified on human mRNAs are classified based on whether the position, sequence context, or ORF type are conserved in the mouse orthologous mRNAs (solid line and dotted line are different type). The TIS site with a mouse counterpart at the identical position or with a similar local sequence context on the aligned orthologous sequences are merged. Both uTIS and dTIS positions are each classified into two subsets according to the global alignment score of sequences (5′UTR for uTIS and CDS for dTIS). Percentage values are presented in the table.

FIGS. 12A-B relate to ORF conservation in ncRNAs. In FIG. 12A, translation in ncRNA SNHG13 is illustrated by LTM and CHX-associated RPF reads. PhastCons scores retrieved from the primate genome sequence alignment is also plotted (bottom panel). In FIG. 12B, translation in ncRNA LOC100128881 is illustrated by LTM and CHX-associated RPF reads. PhastCons scores retrieved from the primate genome sequence alignment is also plotted (bottom panel).

### DETAILED DESCRIPTION OF THE INVENTION

One aspect of the present invention relates to a method for identifying a translation initiation site on an mRNA. This method involves providing a first mRNA in an environment suitable for translation. The first mRNA is contacted with a first translation inhibitor to preferentially stabilize one or more initiation ribosomes at translation initiation sites on the first mRNA. A second mRNA is provided in an environment suitable for translation, where the second mRNA has a nucleotide sequence that is substantially similar to a nucleotide sequence of the first mRNA. The second mRNA is contacted with a second translation inhibitor different from the first translation inhibitor to stabilize one or more initiation ribosomes and one or more elongation ribosomes on the second mRNA. The location of ribosomes stabilized on the first mRNA is compared to the location of ribosomes stabilized on the second mRNA, where ribosomes stabilized at a location on the first mRNA at a higher density than ribosomes stabilized at the same location on the second mRNA identifies the location as a translation initiation site on the first and second mRNAs.

Protein synthesis is a fundamental cellular process that is required for decoding the genome to define proteomes of different cell types in a temporally and spatially controlled manner. It is subject to regulation by a multitude of environmental signals during cell proliferation, differentiation, and apoptosis. The monumental task of faithfully converting the genetic information in the form of linear sequences of mRNA into the corresponding polypeptide chains is accomplished by sophisticated machinery that includes both ribonucleic acids and proteins. Among the four major steps of translation in eukaryotes, initiation, elongation, termination, and recycling of ribosomes, the rate-determining step is initiation, during which mRNA is recruited to the 43S ribosome particle prior to the formation of an 80S ribosome at the initiation codon. Not surprisingly, translation initiation is the primary site of signal integration for translation control.

Translation of mRNA in prokaryotes depends upon the presence of the proper prokaryotic signals which differ from those of eukaryotes. Efficient translation of mRNA in prokaryotes requires a ribosome binding site called the Shine-Dalgarno (“SD”) sequence on the mRNA. This sequence is a short nucleotide sequence of mRNA that is located before the start codon, usually AUG, which encodes the amino-terminal methionine of the protein. The SD sequences are complementary to the 3′-end of the 16S rRNA (ribosomal RNA) and probably promote binding of mRNA to ribosomes by duplexing with the rRNA to allow correct positioning of the ribosome. For a review on maximizing gene expression, see Roberts and Lauer, Methods in Enzymology 68:473 (1979), which is hereby incorporated by reference in its entirety.

The method of the present invention involves identifying translation initiation sites on an mRNA. By “translation initiation site” it is meant a location on an mRNA where an initiation ribosome binds. Other terms used to refer to translation initiation sites are “initiation codons” or “start codons.” In carrying out the method of the present invention, mRNAs are provided in an environment suitable for translation. In one embodiment, mRNA is in a solution containing all necessary components for translation. In another embodiment, mRNA is in a cell or cell mixture.

In yet another embodiment, the first mRNA and the second mRNA may be provided as a population of mRNAs. Thus, for example, the first mRNA may be provided as a population of mRNAs of substantially a single mRNA sequence or a population of mRNAs of many different sequences (e.g., the many different mRNA sequences of a cell).

Suitable mRNAs for carrying out the method of the present invention include, for example, whole-length or fragment mRNAs from a eukaryotic cell, a prokaryotic cell, and/or other sources, such as viruses. When the mNRA is from a virus, it may be from, among others, picornaviruses, flaviviruses, coronaviruses, hepatitis B viruses, rhabdoviruses, adenoviruses, and parainfluenza viruses. Other viruses include polioviruses, rhinoviruses, hepatitis A viruses, coxsackie viruses, encephalomyocarditis viruses, foot-and-mouth disease viruses, echo viruses, hepatitis C viruses, infectious bronchitis viruses, duck hepatitis B viruses, human hepatitis B viruses, vesicular stomatitis viruses, and sendai viruses.

According to the method of the present invention, a first mRNA is contacted with a first translation inhibitor to preferentially stabilize one or more initiation ribosomes at translation initiation sites on the first mRNA. The translation inhibitor may also block translocation of the initiation ribosomes.

The first translation inhibitor preferentially stabilizes initiation ribosomes at translation initiation sites on the first mRNA. As used herein, an “initiation ribosome” refers to a ribosome positioned at a translation initiation site on an mRNA. Because it preferentially stabilizes initiation ribosomes at translation initiation sites on the mRNA most, but not necessarily all, of the ribosomes stabilized on the mRNA by the first translation inhibitor are at translation initiation sites. Thus, according to one embodiment, upon treatment of the first mRNA with the first translation inhibitor, the first translation inhibitor binds one or more ribosomes on translation initiation sites to prevent elongation by the bound initiation ribosome. Ribosomes on the mRNA at elongation sites (i.e., sites other than translation initiation sites) are not bound by the first translation inhibitor and are therefore not stabilized on the first mRNA (i.e., they proceed to “run off” the first mRNA).

The term “stabilize” as used herein with reference to stabilizing a ribosome on a mRNA, means the ribosome is arrested on the mRNA, and is precluded from proceeding with translation. In one embodiment, the ribosome is blocked from translocation.

The first translation inhibitor may preferentially stabilize initiation ribosomes on a mRNA by a variety of mechanisms. In one embodiment, the first translation inhibitor preferentially binds the initiation ribosome after the ribosome is assembled at the translation initiation site. For example, the first translation inhibitor may bind the initiation ribosome in a way to permit the formation of a first peptide bond in translation of the mRNA, but no subsequent peptide bonds.

In one embodiment, the first translation inhibitor is lactimodomycin. Other translation inhibitors that preferentially stabilize ribosomes on translation initiation sites, which are now known or yet to be discovered, may also be used as first translation inhibitors according to the present invention.

According to the method of the present invention, the first mRNA has a nucleotide sequence, and the second mRNA has a nucleotide sequence that is substantially similar to a nucleotide sequence of the first mRNA. According to one embodiment, the nucleotide sequence of the first mRNA, to which the nucleotide sequence of the second mRNA is substantially similar, comprises at least about 25 nucleotides. Alternatively, at least about 50, 75, 100, 150, 200, 250, 300, 350, 400, 450, 500, or more nucleotides constitute the sequence of substantial similarity between the first and second mRNAs.

By “substantially similar” or “substantial similarity,” it is meant that the two sequences have an alignment score, using alignment software known and used by persons of ordinary skill in the art, of at least 70%, 75%, 80%, 85%, 90%, 91%, 92%, 93%, 94%, 95%, 96%, 97%, 98%, 99%, or 100% identity.

In one embodiment, the first mRNA and the second mRNA are simply samples taken from the same population of mRNAs. According to this embodiment, the first mRNA is a population of mRNAs and the second mRNA is a population of mRNAs, and there is little to no difference between the two populations of mRNAs. In other words, the nucleotide sequences of mRNAs in the first population are essentially identical to the nucleotide sequences of mRNAs in the second population.

In other embodiments, it may be useful to compare mRNAs from different individuals of the same species or mRNAs from different species.

The second translation inhibitor is different from the first translation inhibitor in that it stabilizes initiation ribosomes and elongation ribosomes on mRNA. As used herein, “elongation ribosomes” are ribosomes on an mRNA at a position other than a translation initiation site. In one embodiment, the second translation inhibitor does not distinguish between initiation ribosomes or elongation ribosomes, but targets both types of ribosomes.

In one embodiment, the second translation inhibitor is cycloheximide. Other translation inhibitors that do not discriminate between initiation ribosomes and elongation ribosomes, some of which may now be known and other of which are yet to be discovered, may also be used as second translation inhibitors according to the present invention.

The method of the present invention involves comparing the location of ribosomes stabilized on the first mRNA to the location of ribosomes stabilized on the second mRNA. Ribosomes stabilized at a location on the first mRNA at a higher density than ribosomes stabilized at the same location on the second mRNA identifies the location as a translation initiation site on the first and second mRNAs. As mentioned above, the first translation inhibitor preferentially stabilizes initiation ribosomes on translation initiation sites. Thus, in most cases, but not necessarily all cases, the first translation inhibitor will stabilize only initiation ribosomes and therefore, only identify translation initiation sites. The method of the present invention is particularly useful because it involves comparing (i) the location of ribosomes stabilized on the mRNA by the first translation inhibitor, which is preferential to initiation ribosomes to (ii) ribosomes stabilized by the second translation inhibitor which does not distinguish initiation ribosomes from elongation ribosomes. Accordingly, ribosomes stabilized on the same location of an mRNA treated with the first translation inhibitor and the second translation inhibitor identifies that location as a translation initiation site.

When the first and second mRNAs are mRNA populations, a density of ribosomes stabilized at a particular site from the first mRNA population that is equal to or (more likely) greater than a density of ribosomes stabilized at the same site from the second mRNA population identifies that location as a translation initiation site.

As discussed in more detail in the Examples below, translation initiation sites on an mRNA may or may not be an AUG codon. In one embodiment, the translation initiation site is an AUG codon. In another embodiment, the translation initiation site is a codon other than an AUG codon.

The method of the present invention may further involve contacting one or both of the first and second mRNAs with a compound capable of causing dissociation of elongating ribosomes from the first and/or second mRNA. Thus, unlike the first translation inhibitor and the second translation inhibitor, which stabilize a ribosome on an mRNA, other translation inhibitors can cause dissociation of a ribosome from an mRNA. The method of the present invention may further involve contacting the first and/or second mRNA with such a compound.

In one embodiment, the compound capable of causing dissociation of elongating ribosomes from the mRNA is puromycin. Other translation inhibitors that cause dissociation of elongating ribosomes from mRNA, some of which may now be known and others of which are yet to be discovered, may also be used in the method of the present invention.

Another aspect of the present invention relates to a kit for identifying a translation initiation site on an mRNA. The kit includes a first translation inhibitor capable of preferentially stabilizing initiation ribosomes at translation initiation sites on an mRNA. Also included in the kit is a second translation inhibitor different from the first translation inhibitor, where the second translation inhibitor is capable of stabilizing initiation ribosomes and elongation ribosomes on an mRNA. The kit also includes instructions for (i) contacting a first mRNA with the first translation inhibitor and a second mRNA with the second translation inhibitor and (ii) comparing the location of ribosomes stabilized on the first mRNA to ribosomes stabilized on the second mRNA to identify a translation initiation sites on the first and second mRNAs.

### EXAMPLES

The following examples are provided to illustrate embodiments of the present invention but are by no means intended to limit its scope.

### Example 1

### Global Mapping of Translation Initiation Sites in Mammalian Cells at Single-Nucleotide Resolution

Experimental Design

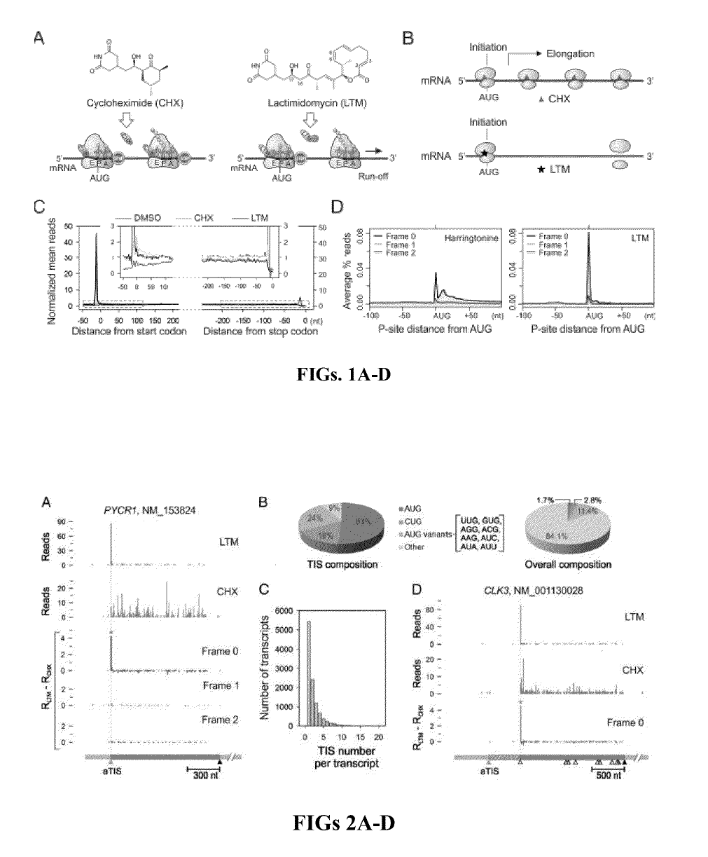

Cycloheximide has been widely used in ribosome profiling of eukaryotic cells because of its potency in stabilizing ribosomes on mRNAs. Both the biochemical (Schneider-Poetsch et al., “Inhibition of Eukaryotic Translation Elongation by Cycloheximide and Lactimidomycin,” Nat. Chem. Biol. 6(3):209-217 (2010), which is hereby incorporated by reference in its entirety) and structural studies (Klinge et al., “Crystal Structure of the Eukaryotic 60S Ribosomal Subunit in Complex with Initiation Factor 6,” Science 334(6058):941-948 (2011), which is hereby incorporated by reference in its entirety) revealed that CHX binds to the exit (E)-site of the large ribosomal subunit, close to the position where the 3′ hydroxyl group of the deacylated tRNA normally binds. CHX thus prevents the release of deacylated tRNA from the (E)-site and blocks subsequent ribosomal translocation (FIG. 1A, left panel). A new family of CHX-like natural products isolated from Streptomyces was recently characterized, including lactimidomycin (Ju et al., “Lactimidomycin, Iso-Migrastatin and Related Glutarimide-Containing 12-Membered Macrolides are Extremely Potent Inhibitors of Cell Migration,” J. Am. Chem. Soc. 131(4):1370-1371 (2009) and Sugawara et al., “Lactimidomycin, a New Glutarimide Group Antibiotic. Production, Isolation, Structure and Biological Activity,” J. Antibiot. (Tokyo) 45(9):1433-1441 (1992), which are hereby incorporated by reference in their entirety). Acting as a potent protein synthesis inhibitor, LTM uses a similar, but not identical, mechanism as CHX (Schneider-Poetsch et al., “Inhibition of Eukaryotic Translation Elongation by Cycloheximide and Lactimidomycin,” Nat. Chem. Biol. 6(3):209-217 (2010), which is hereby incorporated by reference in its entirety). With its 12-membered macrocycle, LTM is significantly larger in size than CHX (FIG. 1A). As a result, LTM cannot bind to the (E)-site when a deacylated tRNA is present. Only during the initiation step, in which the initiator tRNA directly enters into the peptidyl (P)-site (Steitz, “A Structural Understanding of the Dynamic Ribosome Machine,” Nat. Rev. Mol. Cell Biol. 9(3):242-253 (2008), which is hereby incorporated by reference in its entirety), is the empty (E)-site accessible to LTM. Thus, LTM preferentially acts on the initiating ribosome but not the elongating ribosome. It was reasoned that ribosome profiling using LTM side-by-side in comparison with CHX should allow for a complete segregation of the ribosome stalled at the start codon from the one in active elongation (FIG. 1B).

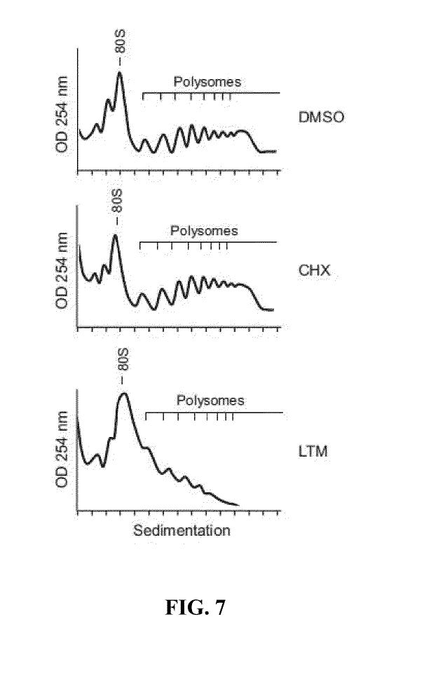

An integrated GTI-seq approach was designed, and ribosome profiling in HEK293 cells pretreated with either LTM or CHX was performed. While CHX slightly stabilized the polysomes when compared to the no-drug treatment (DMSO), 30 min of LTM treatment led to a large increase in monosome accompanied by a depletion of polysomes (FIG. 7). This is in agreement with the notion that LTM halts translation initiation while allowing elongating ribosomes to run off (Schneider-Poetsch et al., “Inhibition of Eukaryotic Translation Elongation by Cycloheximide and Lactimidomycin,” Nat. Chem. Biol. 6(3):209-217 (2010), which is hereby incorporated by reference in its entirety). After RNase I digestion of the ribosome fractions, the purified RPFs were subjected to deep sequencing. As expected, CHX treatment resulted in an excess of RPFs at the beginning of ORFs in addition to the body of CDS (FIG. 1C). Remarkably, LTM treatment led to a pronounced single peak located at the −12 nucleotide (nt) position relative to the annotated start codon. This position corresponds to the ribosome P-site at the AUG codon when an offset of 12 nucleotides is considered (Ingolia et al., “Genome-Wide Analysis In Vivo of Translation with Nucleotide Resolution using Ribosome Profiling,” Science 324(5924):218-223 (2009); and Guo et al., “Mammalian MicroRNAs Predominantly Act to Decrease Target mRNA Levels,” Nature 466(7308):835-840 (2010), which are hereby incorporated by reference in their entirety). LTM treatment also eliminated the excess of ribosomes seen at the stop codon in untreated cells or in the presence of CHX. Therefore, LTM efficiently stalls the 80S ribosome at the start codons.

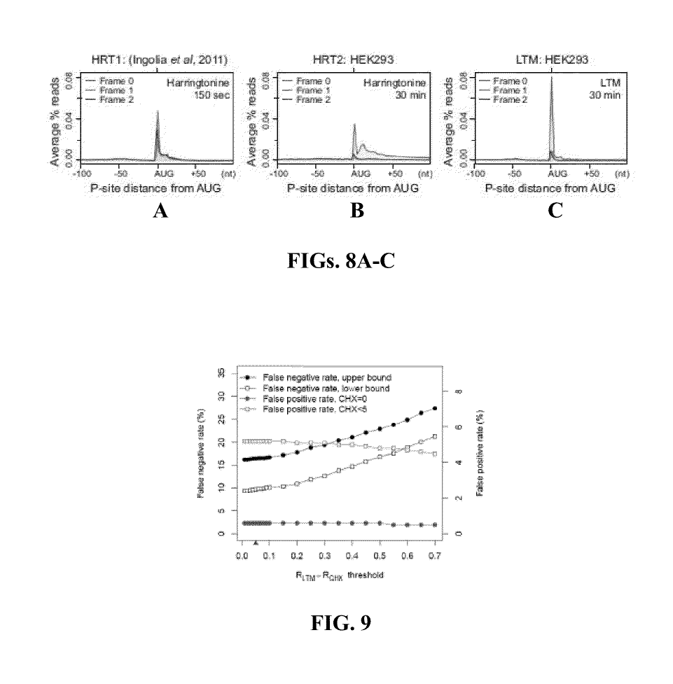

During the course of this study, Ingolia et at reported a similar TIS mapping approach using harringtonine, a different translation initiation inhibitor (Ingolia et al., “Ribosome Profiling of Mouse Embryonic Stem Cells Reveals the Complexity and Dynamics of Mammalian Proteomes,” Cell 147(4):789-802 (2011), which is hereby incorporated by reference in its entirety). One key difference between harringtonine and LTM is that the former drug binds to free 60S subunits (Fresno et al., “Inhibition of Translation in Eukaryotic Systems by Harringtonine,” Eur. J. Biochem. 72(2):323-330 (1977), which is hereby incorporated by reference in its entirety), whereas LTM binds to the 80S complexes already assembled at the start codon (Schneider-Poetsch et al., “Inhibition of Eukaryotic Translation Elongation by Cycloheximide and Lactimidomycin,” Nat. Chem. Biol. 6(3):209-217 (2010), which is hereby incorporated by reference in its entirety). The pattern of RPF density surrounding the annotated start codon was compared between the published datasets (Ingolia et al., “Ribosome Profiling of Mouse Embryonic Stem Cells Reveals the Complexity and Dynamics of Mammalian Proteomes,” Cell 147(4):789-802 (2011), which is hereby incorporated by reference in its entirety) and the LTM results (FIGS. 8A-C). It appears that a considerable amount of harringtonine-associated RPFs are not exactly located at the annotated start codon. To directly compare the TIS mapping accuracy between LTM and harringtonine, ribosome profiling was performed in HEK293 cells treated with harringtonine using the same protocol as the LTM treatment. Similar to the previous study, harringtonine treatment caused a substantial fraction of RPFs accumulated in regions downstream of the start codon (FIG. 1D). The relaxed positioning of harringtonine-associated RPFs after prolonged treatment leaves uncertainty in TIS mapping. In contrast, GTI-seq using LTM largely overcomes this deficiency and offers a high precision in global TIS mapping with single nucleotide resolution (FIG. 1D).

Materials and Methods

HEK293 or MEF cells were treated with 100 μM CHX, 50 μM LTM, 2 μg/ml harringtonine or DMSO at 37° C. for 30 min. Cells were lysed in polysome buffer and cleared lysates were separated by sedimentation through sucrose gradients. Collected polysome fractions were digested with RNase I and the RPF fragments were size selected and purified by gel extraction. After the construction of sequencing library from these fragments, deep sequencing was performed using IlluminaHiSEQ. The trimmed RPF reads with final lengths of 26-29 nt were aligned to the RefSeq transcript sequences by Bowtie-0.12.7 allowing one mismatch. A TIS position on individual transcript was called if the normalized LTM reads density at the every nucleotide position subtracting with that of CHX was well above the background. In analysis of non-coding RNA, only reads unique to single ncRNA were used. To experimentally validate the identified TIS codons, specific genes encompassing both the 5′UTR and the CDS were amplified by RT-PCR from total cellular RNAs extracted from HEK293 cells. The resultant cDNAs were cloned into pcDNA3.1 containing c-myc tag at the COOH-terminus. After transfection into HEK293 cells, whole cell lysates were used for immunoblotting using anti-myc antibody.

Cell Culture and Drug Treatment

Human HEK293 and mouse embryonic fibroblast (“MEF”) were maintained in Dulbecco's Modified Eagle's Medium (“DMEM”) with 10% fetal bovine serum (“FBS”). Cycloheximide was purchased from Sigma and harringtonine from LKT Laboratories. Lactimidomycin was previously described (Ju et al., “Lactimidomycin, Iso-Migrastatin and Related Glutarimide-Containing 12-Membered Macrolides are Extremely Potent Iinhibitors of Cell Migration,” J. Am. Chem. Soc. 131(4):1370-1371 (2009), which is hereby incorporated by reference in its entirety). All drugs were dissolved in DMSO. Cells were treated with 100 μM CHX, 50 μM LTM, 2 μg/ml (3.8 μM) harringtonine or equal volume of DMSO at 37° C. for 30 min.

Polysome Profiling

Sucrose solution was prepared in polysome buffer (pH 7.4, 10 mM HEPES, 100 mM KCl, 5 mM MgCl₂). Sucrose density gradients (15%-45% w/v) were freshly made in SW41 ultracentrifuge tubes (Backman) using a Gradient Master (BioComp Instruments) according to the manufacturer's instructions. Cells were washed using ice-cold PBS containing 100 μg/ml CHX and then lysed by scraping extensively in polysome lysis buffer (pH 7.4, 10 mM HEPES, 100 mM KCl, 5 mM MgCl₂, 100 μg/ml CHX, and 2% Triton X-100). For DMSO control, the CHX was omitted in both PBS and polysome lysis buffer. Cell debris was removed by centrifugation at 14,000 rpm for 10 min at 4° C. 600 μl of supernatant was loaded onto sucrose gradients followed by centrifugation for 100 min at 38,000 rpm and 4° C. in a SW41 rotor. Separated samples were fractionated at 0.750 ml/min through a fractionation system (Isco) that continually monitored OD₂₅₄ values. Fractions were collected at 0.5 min intervals.

Purification of Ribosome Protected mRNA Fragments (RPF)

The general procedure of RPF purification was based on the previously reported protocol (Ingolia et al., “Genome-Wide Analysis In Vivo of Translation With Nucleotide Resolution Using Ribosome Profiling,” Science 324(5924):218-223 (2009), which is hereby incorporated by reference in its entirety) with some modifications. In brief, polysome profiling fractions were mixed and a 140 μl aliquot was digested with 200 U E. coli RNase I (Ambion) at 4° C. for 1 h. Total RNA was then extracted by Trizol reagent (Invitrogen) followed by dephosphorylation with 20 U T4 polynucleotide kinase (NEB) in the presence of 10 U SUPERase_In (Ambion) at 37° C. for 1 hour. The enzyme was heat-inactivated for 20 min at 65° C. The digested RNA products were then separated on a Novex denaturing 15% polyacrylamide TBE-urea gel (Invitrogen). The gel was stained with SYBR Gold (Invitrogen) to visualize the digested RNA fragments. Gel bands around 28 nucleotide RNA molecules were excised and physically disrupted by centrifugation through the holes of the tube. The gel debris was soaked overnight in the RNA gel elution buffer (300 mM NaOAc pH 5.5, 1 mM EDTA, 0.1 U/mL SUPERase_In) to recover the RNA fragments. The gel debris was filtered out with a Spin-X column (Corning) and RNA was purified using ethanol precipitation.

cDNA Library Construction and Deep Sequencing

Poly-A tails were added to the purified RNA fragments by E. coli poly-(A) polymerase (NEB) with 1 mM ATP in the presence of 0.75 U/μL SUPERase_In at 37° C. for 45 min. The tailed RNA molecules were reverse transcribed to generate the first strand cDNA using SuperScript III (Invitrogen) and following oligos containing barcodes:

SCT01:
(SEQ ID NO: 1)
5′-pCTGATCGTCGGACTGTAGAACTCTCAAGCAGAAGACGGCATACGA
TTTTTTTTTTTTTTTTTTTTVN-3′;
MCA02:
(SEQ ID NO: 2)
5′-pCAGATCGTCGGACTGTAGAACTCTCAAGCAGAAGACGGCATACGA
TTTTTTTTTTTTTTTTTTTTVN-3′;
LGT03:
(SEQ ID NO: 3)
5′-pGTGATCGTCGGACTGTAGAACTCTCAAGCAGAAGACGGCATACGA
TTTTTTTTTTTTTTTTTTTTVN-3′;
HTC04:
(SEQ ID NO: 4)
5′-pTCGATCGTCGGACTGTAGAACTCTCAAGCAGAAGACGGCATACGA
TTTTTTTTTTTTTTTTTTTTVN-3′;
and
YAG05:
(SEQ ID NO: 5)
5′-pAGGATCGTCGGACTGTAGAACTCTCAAGCAGAAGACGGCATACGA
TTTTTTTTTTTTTTTTTTTTVN-3′.

Reverse transcription products were resolved on a 10% polyacrylamide TBE-urea gel as described above. The expected 92 nucleotide band of the first strand cDNA was excised and recovered using DNA gel elution buffer (300 mM NaCl, 1 mM EDTA). The purified first strand cDNA was then circularized by 100 U CircLigase II (Epicentre) following the manufacturer's instructions. The circular single strand DNA was purified using ethanol precipitation and re-linearized by 7.5 U APE 1 in 1× buffer 4 (NEB) at 37° C. for 1 h. The linearized products were resolved on a Novex 10% polyacrylamide TBE-urea gel (Invitrogen). The expected 92 nucleotide band was then excised and recovered.

The single-stranded template was then amplified by PCR using the Phusion High-Fidelity enzyme (NEB) according to the manufacturer's instructions. The primers

qNTI200
(SEQ ID NO: 6)
(5′-CAAGCAGAAGACGGCATA-3′)
and
qNTI201
(SEQ ID NO: 7)
(5′-AATGATACGGCGACCACCGACAGGTTCAGAGTTCTACAGTCCGA
CG-3′)

were used to create a DNA library suitable for sequencing. The PCR reaction contained 1×HF buffer, 0.2 mM dNTP, 0.5 μM primers, and 0.5 U Phusion polymerase. PCR was carried out with an initial 30 s denaturation at 98° C., followed by 12 cycles of 10 s denaturation at 98° C., 20 s annealing at 60° C., and 10 s extension at 72° C. PCR products were separated on a non-denaturing 8% polyacrylamide TBE gel as described above. A 120 by band was excised and recovered as described above. After quantification by Agilent BioAnalyzer DNA 1000 assay, equal amounts of barcoded samples were pooled into one sample. 3˜5 pmol mixed DNA samples were typically used for cluster generation followed by sequencing using sequencing primer

(SEQ ID NO: 8)
5′-CGACAGGTTCAGAGTTCTACAGTCCGACGATC-3′
(IlluminaHiSEQ, Cornell University Life Sciences
Core Laboratories Center).

Mapping Ribosome Protected mRNA Fragments to RefSeq Transcripts

To remove adaptor sequences, seven nucleotides were cut off from the 3′ end of each 50 nucleotide-long Illumina sequence read and a stretch of A's were removed from the 3′ end, allowing one mismatch. The remaining insert sequence was separated according to the 2-nucleotide barcode at the 5′ end after the barcode was removed. Reads of length between 26 to 29 nucleotides were mapped to the sense strand of the entire human or mouse RefSeq transcript sequence library (release 49), using Bowtie-0.12.7 (Langmead et al., “Ultrafast and Memory-Efficient Alignment of Short DNA Sequences to the Human Genome,” Genome Biol. 10(3):R25 (2009), which is hereby incorporated by reference in its entirety). Reads mapped to the PhiX genome if any were removed beforehand. One mismatch was allowed in all mappings and in case of multiple mapping, mismatched positions were not used if a perfect match existed. Reads mapped more than 100 times were discarded to remove poly-A-derived reads. Finally, reads were counted at every position of individual transcript by using the 13th nucleotide of the read for the P-site position. Two HEK293 technical replicate controls from the starvation dataset were pooled for most analyses representing HEK293.

Coding Sequence Annotation

The most recent freezes of CCDS (consensus coding sequence) data (Pruitt et al., “The Consensus Coding Sequence (CCDS) Project: Identifying a Common Protein-Coding Gene Set for the Human and Mouse Genomes,” Genome Res. 19(7):1316-1323 (2009), which is hereby incorporated by reference in its entirety) were downloaded from the NCBI ftp site to find annotated translational start and end positions on each mRNA. Each of the CCDS nucleotide sequences were mapped to the associated RefSeq mRNA sequences based on following conditions: (1) the first three nucleotides must be perfectly matched; (2) up to two mismatches are allowed in the first ten nucleotides; (3) up to twenty mismatches are allowed in the full length, with no gaps allowed. The maximum number of mismatches in an accepted alignment was 10.

Read Aggregation Plots

The number of RPF reads aligned to each position of individual transcript was first normalized by the total reads recovered on the same mRNA. The reads counts were then averaged across all mRNAs for each position relative to the annotated start codon. To avoid multiple counting of the same reads mapped to multiple isoforms of the same gene, redundant mRNAs were removed based on the sequence context of −100 nt˜+100 nt relative to the annotated TIS. The same approach was used to obtain average read aggregation relative to dTIS or uTIS positions.

Identification of TIS Positions

A peak is defined at the nucleotide level on a transcript. A peak position satisfies the following conditions: (1) the transcript must have both LTM and CHX reads; (2) the position must have at least 10 reads from the LTM data; (3) the position must be a local maximum within 7 nucleotides; (4) the position must have “LTM-CHX”=(X_{LTM}/N_{LTM}−X_{CHX}/N_{CHX}) to be at least 0.005, where Xₖ is the number of reads on that position in data k and Nₖ is the total number of reads on that transcript in data k. Generally, a peak position is also called a ‘TIS’. However, if a peak was not detected on the first position of any AUG or near-cognate start codon but was present at the first position of an immediately preceding or succeeding one of these codons, the position was called a TIS.

Identification of Potentially Misannotated aTIS

Among mRNAs with at least one identified dTIS position, those with no aTIS or uTIS peak were selected. Then, the first dTIS in frame 0 was identified as the potentially correct aTIS (pcaTIS). If this dTIS was not associated with an AUG or near-cognate start codon, it was discarded. Any mRNA with a 5′UTR shorter than 12 nucleotides is excluded, because the method requires at least a 12 nucleotide 5′UTR in order to detect the aTIS that would be at the 13^{th} position on a read. To reduce possible false positives, it was ensured that: (1) the total CHX reads in the region from position 1 to pcaTIS position −2 on an mRNA was less than 10; (2) the maximum CHX reads in this region was less than 2; (3) total LTM reads from position aTIS−1 to aTIS+1 was 0; and (4) the average CHX read density between pcaTIS−1 and pcaTIS+11 was higher than 0.1 reads per nucleotide.

Codon Composition Analysis

The number of TIS positions associated with each codon type starting was counted. The enumeration was done after filtering redundant TIS positions based on its flanking sequence context from −30 to +122 nucleotides relative to the TIS position to avoid double counting of the TIS on the common regions of transcript iso forms. The same redundancy filtering was applied in most other analyses and counting was as described below. Background codon composition was based on all codons in either annotated CDS or 5′UTR of all mRNAs, regardless of reading frame. Redundancy filtering was not performed for background counting.

Ribosomal Leaky Scanning Analysis

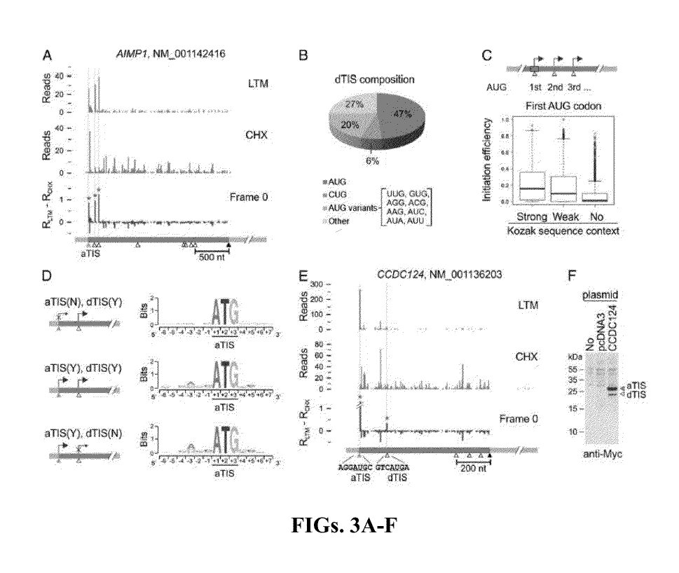

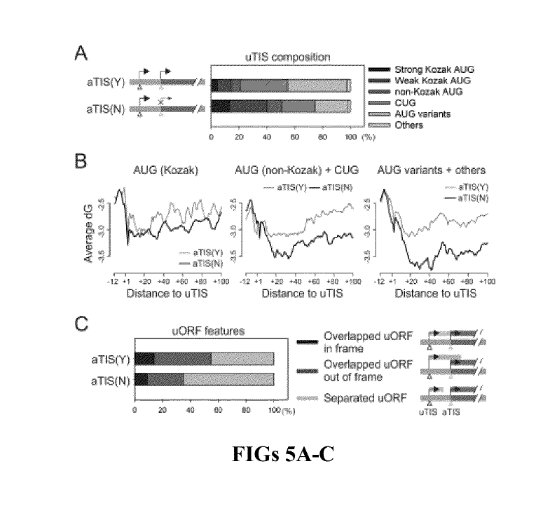

Three subsets of aTIS positions were collected based on whether the aTIS has the initiation peak and whether the mRNA has any detectable AUG-associated dTIS (FIG. 3D). Sequence logos were drawn using Berkeley Weblogo (Crooks et al., “WebLogo: A Sequence Logo Generator,” Genome Res. 14(6):1188-1190 (2004), which is hereby incorporated by reference in its entirety). The uTIS positions with the maximum peak height on an mRNA were grouped according to whether the aTIS has a peak [aTIS(Y)] or not [aTIS(N)] and their Kozak sequence context was analyzed (FIG. 5A). For counting the types of uTIS-associated uORFs (FIG. 5C), the most downstream uTIS on each mRNA was assigned to one of two groups according to whether the aTIS has a peak [aTIS(Y)] or not [aTIS(N)]. The same uTIS sets collected for the Kozak sequence context analysis were used for measurement of free energy of downstream RNA secondary structures. Each of these subsets was divided into three groups according to the initiation context—“AUG (Kozak),” “AUG (non-Kozak)+CUG,” and “AUG variants+others.” The AUG (Kozak) group includes an AUG with either or both of −3A/G and +4G. AUG (non-Kozak) group is an AUG with neither −3A/G nor +4G. For each TIS position, a window length of 22 nt was moved at a step size of 1 nucleotide, starting from −12 nucleotides relative to each uTIS to +100 nucleotide, and the AG was calculated for each window using the RNAfold program (Gruber et al., “The Vienna RNA Websuite,” Nucleic Acids Res. 36(Web Server issue):W70-74 (2008), which is hereby incorporated by reference in its entirety). The AG values were averaged for each position relative to the uTIS across all uTIS positions in each set.

TIS Conservation Between Human and Mouse

Human and mouse RefSeq protein accessions were extracted from HomoloGene (release 65) (Sayers et al., “Database Resources of the National Center for Biotechnology Information,” Nucleic Acids Res. 39(Database issue):D38-51 (2011), which is hereby incorporated by reference in its entirety). Each RefSeq protein accession was matched to the associated mRNA accession, CCDS ID, and CCDS amino acid sequence. The amino acid sequence of each homologous protein pair were aligned to each other using Clustalw 2.1 (Larkin et al., “Clustal W and Clustal X Version 2.0,” Bioinformatics 23(21):2947-2948 (2007), which is hereby incorporated by reference in its entirety), to calculate the alignment score and filter one-to-one orthologous relationships. If two or more proteins from the same species were in the same HomoloGene group, only the single reciprocally best matched pair was used. Likewise, if an orthologous gene has mRNA iso forms, the reciprocally best matched iso form pair was chosen. Any tied matches were removed. The alignment score was computed as [1−(the number of mismatches and gaps)/(length of human protein)]*100. Any alignment with an alignment score less than 50 was discarded. The 5′UTR of an orthologous mRNA was considered as an orthologous 5′UTR.

Among the human mRNAs that have a mouse ortholog, 5′UTRs and CDSs were independently grouped into well-aligned and poorly-aligned categories. A 5′UTR with an alignment score less than 50 or with a 30 nucleotide or longer 3′ end gap is considered poorly aligned. Likewise, a CDS with a 30 nucleotide or longer initial gap is also considered poorly aligned. Note that a CDS with an alignment score less than 50 was discarded beforehand. Within each category, human uTIS or dTIS were classified into five groups, according to sequence conservation (S0 vs S1) and subtype conservation (T0 vs T1).

A TIS is conserved in sequence (S1) if there is a mouse TIS peak at the same position on the aligned orthologous mouse sequence or if there is a mouse TIS peak with a similar surrounding sequence. The surrounding sequence is taken from −6 to +24 nucleotides relative to each uTIS. The sequence similarity must be at least 75% identity with no gaps. If a mouse TIS exist in the orthologous 5′UTR or CDS, but not conserved in sequence, it was assigned to the S0 category. If no mouse TIS existed, it was classified as “N.” If the mouse ortholog had no detectable TIS at all, the pair was removed from the analysis.

A TIS is conserved in subtype (T1) if the corresponding mouse uTIS or dTIS is of the same type. For auTIS, two subtypes, “N-terminal extended” versus “overlapped” and “separated” were considered. For a dTIS, frame 0 versus frame 1 and 2 were used as two subtypes. The priority is set in the order of T1S1, T1S0, T0S1, T0S0, and N, in case a TIS belongs to two or more classes.

Identification of Translated ORFs in Non-Coding RNA and Conservation Analysis

Human and mouse ncRNAs were collected from the RefSeq (release 49) by extracting the RNAs with an accession beginning with “NR” and with no mRNA isoforms. To avoid false detection of TIS positions due to spurious mapping of reads sourced from mRNA transcripts, only reads unique to a single ncRNA were used. From the human ncRNAs with at least one identified TIS, PhastCons score for every nucleotide position within either ORF or non-ORF regions was collected. The PhastCons scores were obtained by using the UCSC Table Browser (http://genome.ucsc.edu) (Karolchik et al., “The UCSC Table Browser Data Retrieval Tool,” Nucleic Acids Res. 32(Database issue):D493-496 (2004) and Kent et al., “The Human Genome Browser at UCSC,” Genome Res. 12(6):996-1006 (2002), which are hereby incorporated by reference in their entirety), from the placental and primate subsets of the 46-way vertebrate genomic alignment. The ncRNAs whose genomic positions were ambiguous (e.g., the ncRNA is not included in the refGene table of the UCSC database or the length of the RNA is different from the refGene record) were excluded from the analysis.

Plasmid Construction and Immunoblotting

cDNA was synthesized by Superscript III RT (Invitrogen) using 1 μg of total RNA extracted from HEK293 cells. CCDCl24 and RND3 gene encompassing both the 5′UTR and the CDS were amplified by PCR reaction using the following oligo pairs:

ccdc124F:
(SEQ ID NO: 9)
5′-GGCGCCAAGCTTGGAGGCGCGACCGGGCCGGCGCTGG-3′;
ccdc124R:
(SEQ ID NO: 10)
5′-GGCGCCCTCGAGTTGGGGGCATTGAAGGGCACGGCCC-3′;
rnd3F:
(SEQ ID NO: 11)
5′-GGCGCCAAGCTTCAGTCGGCTCGGAATTGGACTTGGG-3′;
and
rnd3R:
(SEQ ID NO: 12)
5′-GGCGCCCTCGAGCTATTCTGCACCCTGGAGGCGTAGC-3′.

The PCR fragments were cloned to Hind III and Xho I sites of pcDNA™3.1/myc-His B. Plasmid transfection was performed using Lipofectamine 2000 (Invitrogen) according to the manufacturer's instructions. After 48 hr transfection, cells were lysed by the lysis buffer (Tris-buffered saline, pH 7.4, 2% Triton X-100). The whole cell lysates were heat-denatured for 10 min in NuPAGE® LDS Sample Buffer (Invitrogen). The protein samples were resolved on 12% NuPAGE gel (Invitrogen) and then transferred to Immobilon-P membranes (Millipore). After blocking for 1 hour in TBS containing 5% blotting milk, membranes were incubated with c-myc antibodies (Santa Cruz Biotechnology) at 4° C. overnight. After incubation with horseradish peroxidase-coupled secondary antibodies (Sigma), immunoblots were developed using enhanced chemiluminescence (GE Healthcare).

Global TIS Identification by GTI-seq

One of the advantages of GTI-seq is its ability to analyze LTM data in parallel with CHX. Due to the structural similarity between these two translation inhibitors, the LTM background reads resembled the pattern of CHX-associated RPFs (FIG. 2A). This feature allows one to further reduce the background noise of LTM-associated RPFs by subtracting the normalized CHX reads density at every nucleotide position from that of LTM. A TIS peak is then called at a position in which the adjusted LTM reads density is well above the background (FIG. 2A, asterisk). From ˜4,000 transcripts with detectable TIS peaks, a total of 16,231 TIS sites were identified. Codon composition analysis revealed that more than half of the TIS codons used AUG as the translation initiator (FIG. 2B). GTI-seq also identified a significant proportion of TIS codons employing near-cognate codons that differ from AUG by a single nucleotide, in particular CUG (16%). Remarkably, nearly half of the transcripts (42%) contained multiple TIS sites (FIG. 2C), suggesting that alternative translation prevails even under physiological conditions. Surprisingly, about a third of the transcripts (32.4%) showed no TIS peaks at the annotated TIS position (“aTIS”) despite clear evidence of translation. While some of them could be false negatives due to stringent threshold cutoff for TIS identification (FIG. 9), others were likely attributed to alternative translation initiation (see below). However, it is also possible that some cases represent mis-annotation. For instance, the translation of CLK3 clearly starts from the second AUG, although the first one was annotated as the initiator in the current database (FIG. 2D). 50 transcripts were found to have possible mis-annotation in their start codons. However, it is possible that some mRNAs might have alternative transcript processing. In addition, the possibility that some of these genes might have tissue-specific translation initiation sites could not be excluded.

Characterization of Downstream Initiators

In addition to validating initiation at the annotated start codon, GTI-seq revealed clear evidence of downstream initiation on 39% of the analyzed transcripts with TIS peaks. As a typical example, AIMP1 showed three TIS peaks exactly at the first three AUG codons in the same reading frame (FIG. 3A). Thus, the same transcript generates three isoforms of AIMP1 with varied NH₂-terminus, which is consistent with the previous report (Shalak et al., “Translation Initiation from Two In-Frame AUGs Generates Mitochondrial and Cytoplasmic Forms of the p43 Component of the Multisynthetase Complex,” Biochemistry 48(42):9959-9968 (2009), which is hereby incorporated by reference in its entirety). Of the total TIS positions identified by GTI-seq, 23% (3,741/16,231) were located downstream of aTIS codons, which were termed dTIS, and nearly half of the identified dTIS codons utilized AUG as the initiator (FIG. 3B).

Regarding possible factors influencing downstream start codon selection, genes were classified with multiple TIS codons into three groups based on Kozak consensus sequence of the first AUG. The relative leakiness of the first AUG codon was estimated by measuring the fraction of LTM reads at the first AUG over the total reads recovered on and after this position. The AUG codon with a strong Kozak sequence context showed the highest initiation efficiency (or lowest leakiness) in comparison to the one with weak or no consensus sequence (FIG. 3C, p=1.12×10⁻¹⁴²). These results indicate the important role of sequence context in start codon recognition. To substantiate this conclusion further, a reciprocal analysis was performed by grouping genes according to whether an initiation peak was identified at the aTIS or dTIS positions on their transcripts (FIG. 3D). A survey of the sequences flanking the aTIS revealed a clear preference of Kozak sequence context for different gene groups. In the gene group with aTIS initiation, but no detectable dTIS, the strongest Kozak consensus sequence was observed (FIG. 3D, bottom panel). This sequence context was largely absent in the group of genes lacking detectable translation initiation at the aTIS (FIG. 3D, top panel). Thus, ribosome leaky scanning tends to occur when the context of an aTIS is suboptimal.

Cells use the leaky scanning mechanism to generate protein iso forms with changed subcellular localizations or altered functionality from the same transcript (Kochetov, “Alternative Translation Start Sites and Hidden Coding Potential of Eukaryotic mRNAs,” Bioessays 30(7):683-691 (2008), which is hereby incorporated by reference in its entirety). In addition to genes that have been reported to produce protein iso forms via leaky scanning, GTI-seq revealed many more cases than previously reported. To independently validate the novel dTIS positions identified by GTI-seq, the gene CCDCl24, whose transcript showed several initiation peaks above the background, was cloned (FIG. 3E). One dTIS is in the same reading frame of the aTIS, which allows us to use a COOH-terminal tag to detect different translational products in transfected cells. Immunoblotting of transfected HEK293 cells showed two clear bands whose molecular weights correspond to the full length of CCDCl24 (28.9 kDa) and the NH₂-terminally truncated iso form (23.7 kDa), respectively. The relative abundance of both isoforms matched well to the corresponding LTM reads density, suggesting that GTI-seq might provide quantitative aspects of translation initiation.

Characterization of Upstream Initiators

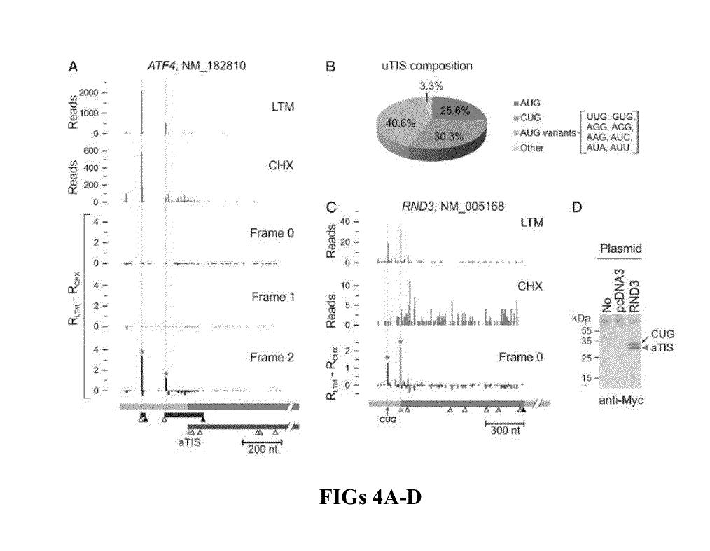

Sequence-based computational analyses predicted that about 50% of mammalian transcripts contain at least one uORF (Calvo et al., “Upstream Open Reading Frames Cause Widespread Reduction of Protein Expression and are Polymorphic Among Humans,” Proc. Natl. Acad. Sci. U.S.A. 106(18):7507-7512 (2009) and Resch et al., “Evolution of Alternative and Constitutive Regions of Mammalian 5′UTRs,” BMC Genomics 10:162 (2009), which are hereby incorporated by reference in their entirety). In agreement with this notion, GTI-seq revealed 54% of transcripts bearing one or more TIS positions upstream of the annotated start codon. These upstream TIS (uTIS) codons, when out of the aTIS reading frame, are often associated with short ORFs. A classic example is ATF4, whose translation is predominantly controlled by several uORFs (Spriggs et al., “Translational Regulation of Gene Expression During Conditions of Cell Stress,” Mol. Cell 40(2):228-237 (2010); Harding et al., “Transcriptional and Translational Control in the Mammalian Unfolded Protein Response,” Annu. Rev. Cell Dev. Biol. 18:575-599 (2002); and Vattem et al., “Reinitiation Involving Upstream ORFs Regulates ATF4 mRNA Translation in Mammalian Cells,” Proc. Natl. Acad. Sci. U.S.A. 101(31):11269-11274 (2004), which are hereby incorporated by reference in their entirety). This feature was clearly captured by GTI-seq (FIG. 4A). In addition to the two known uORFs proximal to the aTIS, another extremely short uORF was identified at the beginning of the ATF4 mRNA. Intriguingly, the AUG start codon is immediately followed by a UAG stop codon. This one-codon uORF was clearly marked by both LTM and CHX-associated RPFs. As expected, the presence of these uORFs efficiently repressed the initiation at the aTIS as evidenced by few CHX reads along the CDS of ATF4. Despite the low enrichment of LTM reads at the aTIS of ATF4, a specific LTM peak was still distinguishable above the background (FIG. 4A). This example highlights the remarkable sensitivity of GTI-seq in capturing TIS codons with low initiation efficiency.

Of the total TIS positions identified by GTI-seq, nearly half of them were uTIS (7,936/16,231). In contrast to the dTIS, which utilized AUG as the primary start codon (FIG. 3B), the majority of uTIS (74.4%) were non-AUG codons (FIG. 4B). Among these AUG variants, CUG was the most prominent one with the frequency even higher than AUG (30.3% vs. 25.6%). In a few well-documented examples, the CUG triplet was reported to serve as an alternative initiator (Touriol et al., “Generation of Protein Isoform Diversity by Alternative Initiation of Translation at Non-AUG Codons,” Biol. Cell 95(3-4):169-178 (2003), which is hereby incorporated by reference in its entirety). To experimentally confirm the alternative initiators identified by GTI-seq, the gene RND3 was cloned and showed a clear initiation peak at a CUG codon in addition to the aTIS (FIG. 4C). The two initiators are in the same reading frame without a stop codon in between, which permits the detection of different translational products using an antibody against the fused COOH-terminal tag. Immunoblotting of transfected HEK293 cells showed two protein bands corresponding to the CUG-initiated long iso form (34 kDa) and the main product (31 kDa) (FIG. 4C). Once again, the levels of both isoforms were in accordance with the relative densities of LTM reads, further supporting the quantitative feature of GTI-seq in TIS mapping.

Global Impacts of uORFs on Translational Efficiency

Initiation from anuTIS, and the subsequent translation of the short uORF, negatively influences the main ORF translation (Morris et al., “Upstream Open Reading Frames as Regulators of mRNA Translation,” Mol. Cell Biol. 20(23):8635-8642 (2000) and Calvo et al., “Upstream Open Reading Frames Cause Widespread Reduction of Protein Expression and are Polymorphic Among Humans,” Proc. Natl. Acad. Sci. U.S.A. 106(18):7507-7512 (2009), which are hereby incorporated by reference in their entirety). To find possible factors governing the alternative TIS selection in the 5′UTR, uTIS-bearing transcripts were categorized into two groups according to whether initiation occurs at the aTIS and compared the sequence context of uTIS codons (FIG. 5A). For transcripts with initiation at both uTIS and aTIS positions [aTIS(Y)], the uTIS codons were preferentially composed of non-optimal AUG variants. In contrast, the uTIS codons identified on transcripts with repressed aTIS initiation [aTIS(N)] showed a higher percentage of AUG with Kozak consensus sequences (p=1.74×10⁻⁸⁰). These results are in agreement with the notion that the accessibility of an aTIS to the ribosome for initiation depends on the context of uTIS codons.

Recent work showed a correlation between secondary structure stability of local mRNA sequences near the start codon and mRNA translation efficiency (Kudla et al., “Coding-Sequence Determinants of Gene Expression in Escherichia coli,” Science 324(5924):255-258 (2009); Kochetov et al., “AUG hairpin: Prediction of a Downstream Secondary Structure Influencing the Recognition of a Translation Start Site,” BMC Bioinformatics 8:318 (2007); and Kertesz et al., “Genome-Wide Measurement of RNA Secondary Structure in Yeast,” Nature 467(7311):103-107 (2010), which are hereby incorporated by reference in their entirety). To examine whether the uTIS initiation is also influenced by local mRNA structures, the free energy associated with secondary structures from regions surrounding the uTIS position was computed (FIG. 5B). An increased folding stability of the region shortly after the uTIS in transcripts with repressed aTIS initiation was observed (FIG. 5B, black line). In particular, more stable mRNA secondary structures were present on transcripts with less optimal uTIS codons (FIG. 5B, right panels). Therefore, when the consensus sequence is absent from the start codon, the local mRNA secondary structure has a stronger correlation with the TIS selection.

Depending on the uTIS positions, the associated uORF can be separated from or overlapped with the main ORF. These different types of uORF could use different mechanisms to control the main ORF translation. For instance, when the uORF is short and separated from the main ORF, the 40S subunit can remain associated to the mRNA after termination at the uORF stop codon and resumes scanning, a process called reinitiation (Jackson et al., “The Mechanism of Eukaryotic Translation Initiation and Principles of its Regulation,” Nat. Rev. Mol. Cell Biol. 11(2):113-127 (2010), which is hereby incorporated by reference in its entirety). When the uORF overlaps with the main ORF, the aTIS initiation solely relies on the leaky scanning mechanism. It was sought to dissect the respective contributions of reinitiation and leaky scanning to the regulation of aTIS initiation. Interestingly, a higher percentage of separated uORFs was found in transcripts with repressed aTIS initiation [aTIS(N) group] (FIG. 5C, p=3.52×10⁻⁴¹). This result suggests that the re-initiation is generally less efficient than leaky scanning, which is consistent with the negative role of uORFs in translation of main ORFs.

Cross-Species Conservation of Alternative Translation Initiators

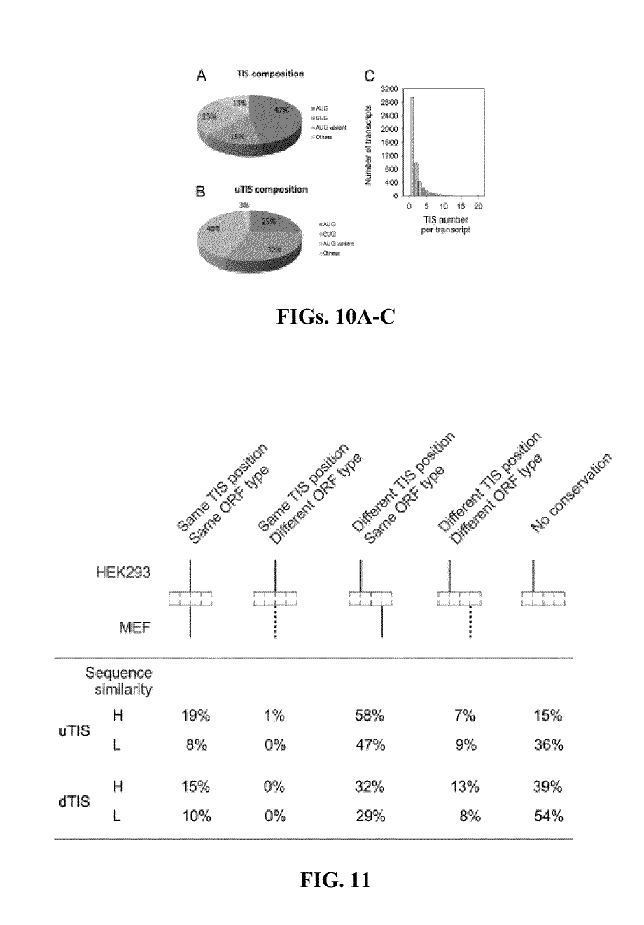

The prevalence of alternative translation re-shapes the proteome landscape by either increasing the protein diversity or modulating translation efficiency. The biological significance of alternative initiators could be preserved across species if they are of potential fitness benefit. GTI-seq was applied to a mouse embryonic fibroblast (“MEF”) cell line and TIS positions were identified across the mouse transcriptome, including uTIS and dTIS. Compared to HEK293 cells, MEF cells showed remarkable similarity in overall TIS features (FIGS. 10A-C). For example, uTIS codons utilized non-AUG, especially CUG, as the dominant initiator. Additionally, about half of the transcripts in MEF cells exhibited multiple initiators. Thus, the general features of alternative translation are well conserved between human and mouse cells.

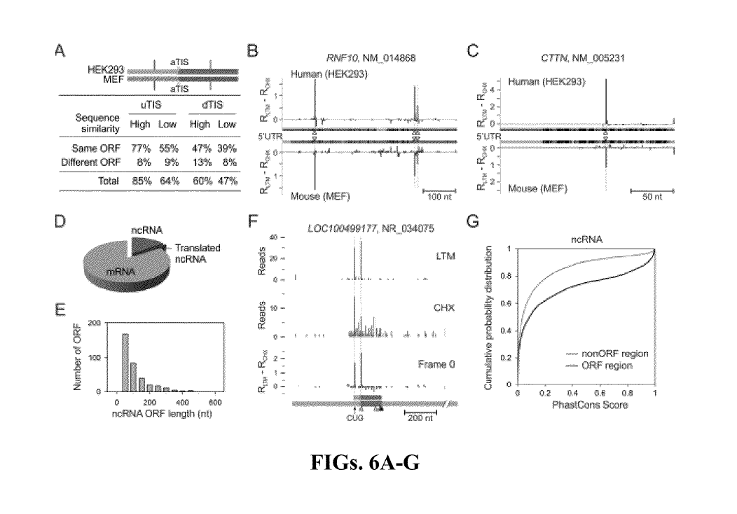

To analyze conservation of individual alternative TIS position on each transcript, a total of 12,949 human-mouse orthologous mRNA pairs were chosen. The 5′UTR and CDS regions were analyzed separately in order to measure the conservation of uTIS and dTIS positions, respectively (FIG. 6A). Each group was classified into two subgroups based on their sequence similarity. For genes with high sequence similarity, 85% of the uTIS and 60% of dTIS positions were conserved between human and mouse cells. Some of these alternative TIS codons were located at the same positions on the aligned sequences (FIG. 11). As an example, RNF10 in HEK293 cells showed three uTIS positions, which were also found in MEF cells at the identical positions on the aligned 5′UTR sequence of the mouse homolog (FIG. 6B). Remarkably, genes with low sequence similarity also displayed high TIS conservation across the two species (FIG. 6A). For instance, the 5′UTR of CTTN gene has low sequence identity between human and mouse homo logs (alignment score=40.3) (FIG. 6C). However, a clear uTIS was identified in both cells at the same position on the aligned region. Notably, the majority of alternative ORFs conserved between human and mouse cells were of the same type, i.e., either separated from or overlapped with the main ORF (FIG. 6A and FIG. 11). The evolutionary conservation of those TIS positions and the associated ORFs is a strong indication of functional significance of alternative translation in the regulation of gene expression.

Characterization of ncRNA Translation

The mammalian transcriptome contains many non-protein-coding RNAs (ncRNAs) (Mattick, “The Functional Genomics of Noncoding RNA,” Science 309(5740):1527-1528 (2005), which is hereby incorporated by reference in its entirety). ncRNAs have gained much attention recently due to their emerging role in a variety of cellular processes including embryogenesis and development (Pauli et al., “Non-Coding RNAs as Regulators of Embryogenesis,” Nat. Rev. Genet. 12(2):136-149 (2011), which is hereby incorporated by reference in its entirety). Motivated by the recent report about the possible translation of large intergenic ncRNAs (lincRNAs) (Ingo lia et al., “Ribosome Profiling of Mouse Embryonic Stem Cells Reveals the Complexity and Dynamics of Mammalian Proteomes,” Cell 147(4):789-802 (2011), which is hereby incorporated by reference in its entirety), the possible translation, or at least ribosome association, of ncRNAs was explored in HEK293 cells. RPFs uniquely mapped to ncRNA sequences were selected to exclude the possibility of spurious mapping of reads originated from mRNAs. Of 5,763 ncRNAs annotated in RefSeq, 169 ncRNAs (about 3%) were identified that were associated with RPFs marked by both CHX and LTM (FIG. 6D). Compared to protein-coding mRNAs, most ORFs recovered from ncRNAs were very short with a median length of 82 nucleotides (FIG. 6E). Several ncRNAs also showed alternative initiation at non-AUG start codons as exemplified by LOC100499177 (FIG. 6F).

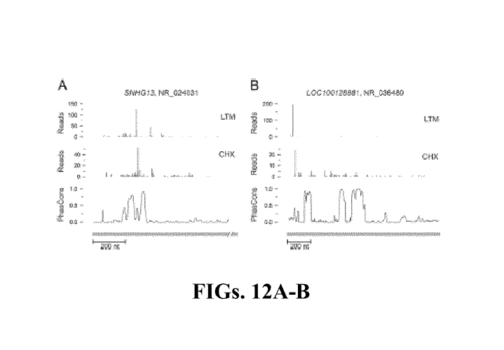

Comparative genomics reveals that the coding regions are often evolutionarily conserved elements (Siepel et al., “Evolutionarily Conserved Elements in Vertebrate, Insect, Worm, and Yeast Genomes,” Genome Res. 15(8):1034-1050 (2005), which is hereby incorporated by reference in its entirety). The PhastCons scores for both coding and non-coding regions of ncRNAs were retrieved and it was found that the ORF regions identified by GTI-seq indeed showed a higher conservation (FIG. 6G). Some ncRNAs showed a clear enrichment of highly conserved bases within the ORFs marked by both LTM and CHX reads (FIGS. 12A-B). Despite the apparent engagement by the protein synthesis machinery, the physiological functions of the coding capacity of these ncRNAs remain to be determined.

Discussion

The mechanisms of eukaryotic translation initiation have received increasing attention owing to their central importance in diverse biological processes (Sonenberg et al., “Regulation of Translation Initiation in Eukaryotes: Mechanisms and Biological Targets,” Cell 136(4):731-745 (2009), which is hereby incorporated by reference in its entirety). The use of multiple initiation codons in a single mRNA contributes to protein diversity by expressing several protein isoforms from a single transcript. Distinct ORFs defined by alternative TIS codons could also serve as regulatory elements in controlling the translation of the main ORF (Morris et al., “Upstream Open Reading Frames as Regulators of mRNA Translation,” Mol. Cell Biol. 20(23):8635-8642 (2000) and Calvo et al., “Upstream Open Reading Frames Cause Widespread Reduction of Protein Expression and are Polymorphic Among Humans,” Proc. Natl. Acad. Sci. U.S.A. 106(18):7507-7512 (2009), which are hereby incorporated by reference in their entirety). Although there is some understanding of how ribosomes determine where and when to start initiation, the knowledge is far from complete. GTI-seq provides a comprehensive and high-resolution view of TIS positions across the entire transcriptome. The precise TIS mapping offers mechanistic insights into the start codon recognition.

Global TIS Mapping at Single Nucleotide Resolution by GTI-seq

Traditional toeprinting analysis showed heavy ribosome pausing at both the initiation and the termination codons of mRNAs (Wolin et al., “Signal Recognition Particle Mediates a Transient Elongation Arrest of Preprolactin in Reticulocyte Lysate,” J. Cell Biol. 109(6 Pt 1):2617-2622 (1989) and Sachs et al., “Toeprint Analysis of the Positioning of Translation Apparatus Components at Initiation and Termination Codons of Fungal mRNAs,” Methods 26(2):105-114 (2002), which are hereby incorporated by reference in their entirety). Consistently, deep sequencing-based ribosome profiling also revealed the highest RPF density at both the start and the stop codons (Ingolia et al., “Genome-Wide Analysis In Vivo of Translation with Nucleotide Resolution using Ribosome Profiling,” Science 324(5924):218-223 (2009) and Guo et al., “Mammalian MicroRNAs Predominantly Act to Decrease Target mRNA Levels,” Nature 466(7308):835-840 (2010), which are hereby incorporated by reference in their entirety). Although this feature enables approximate determination of decoded mRNA regions, it does not allow for unambiguous identification of TIS positions especially when multiple initiators are utilized. Translation inhibitors specifically acting on the first round of peptide bond formation allow the run-off of elongating ribosomes, thereby specifically halting ribosomes at the initiation codon. Indeed, harringtonine treatment caused a profound accumulation of RPFs in the beginning of CDS (Ingolia et al., “Ribosome Profiling of Mouse Embryonic Stem Cells Reveals the Complexity and Dynamics of Mammalian Proteomes,” Cell 147(4):789-802 (2011), which is hereby incorporated by reference in its entirety). A caveat of using harringtonine is that this drug binds to free 60S subunits and the inhibitory mechanism is unclear. In particular, it is not known whether harringtonine completely blocks the initiation step. It was observed that a significant fraction of ribosomes still passed over the start codon in the presence of harringtonine.

The translation inhibitor L™ bears several features in achieving the high resolution of global TIS identification. First, LTM binds to the 80S ribosome already assembled at the initiation codon and permits the first peptide bond formation (Schneider-Poetsch et al., “Inhibition of Eukaryotic Translation Elongation by Cycloheximide and Lactimidomycin,” Nat. Chem. Biol. 6(3):209-217 (2010), which is hereby incorporated by reference in its entirety). Thus, the LTM-associated RPF more likely represents physiological TIS positions. Second, LTM occupies the empty E-site of initiating ribosomes and thus completely blocks the translocation. This feature allows the TIS identification at single nucleotide resolution. With this precision, different reading frames become unambiguous, thereby revealing different types of ORFs within each transcript. Third, owing to the similar structure and the same binding site in the ribosome, LTM and CHX can be applied side-by-side to achieve simultaneous assessment of both initiation and elongation for the same transcript. With the high signal/noise ratio, GTI-seq offers a direct TIS identification approach with a minimal computational aid. The uncovering of alternative initiators allows probing of mechanistic insights of TIS selection. Also, different translational products initiated from alternative start codons, including non-AUG, can be experimentally validated. Further confirming the accuracy of GTI-seq, a sizable fraction of alternative start codons identified by GTI-seq exhibited high conservation across species. The evolutionary conservation strongly suggests a physiological significance of alternative translation in gene expression.

Diversity and Complexity of Alternative Start Codons

GTI-seq revealed that the majority of identified TIS positions belong to alternative start codons. The prevailing alternative translation was corroborated by the finding that nearly half of the transcripts contained multiple TIS codons. While dTIS codons use the conventional AUG as the main initiator, a significant fraction of uTIS codons are non-AUG with the CUG as the most frequent one. In a few well-documented cases, including FGF2 (Vagner et al., “Translation of CUG- but Not AUG-Initiated Forms of Human Fibroblast Growth Factor 2 is Activated in Transformed and Stressed Cells,” J. Cell Biol. 135(5):1391-1402 (1996), which is hereby incorporated by reference in its entirety), VEGF (Meiron et al., “New Iso forms of VEGF are Translated from Alternative Initiation CUG Codons Located in its 5′UTR,” Biochem. Biophys. Res. Commun. 282(4):1053-1060 (2001), which is hereby incorporated by reference in its entirety), and Myc (Hann et al., “A Non-AUG Translational Initiation in c-myc Exon 1 Generates an N-Terminally Distinct Protein Whose Synthesis is Disrupted in Burkitt's Lymphomas,” Cell 52(2):185-195 (1988), which is hereby incorporated by reference in its entirety), the CUG triplet was reported to serve as the non-AUG start codon. With the high resolution TIS map across the entire transcriptome, GTI-seq greatly expanded the list of hidden coding potential of mRNAs not visible by sequence-based in silico analysis.

GTI-seq revealed several lines of evidence supporting the linear scanning mechanism for start codon selection. First, the uTIS context, such as the Kozak consensus sequence and the secondary structure, largely influenced the frequency of aTIS initiation. Second, the stringency of an aTIS codon negatively regulated the dTIS efficiency. Third, the leaky potential at the first AUG was inversely correlated with the strength of its sequence context. Since it is less likely for a preinitiation complex to bypass a strong initiator to select a downstream suboptimal one, it is not surprising that most uTIS codons are not canonical, whereas the dTIS codons are mostly conventional AUG. In addition to the leaky scanning mechanism for alternative translation initiation, ribosomes could translate a short uORF and reinitiate at downstream ORFs (Jackson et al., “The Mechanism of Eukaryotic Translation Initiation and Principles of its Regulation,” Nat. Rev. Mol. Cell Biol. 11(2):113-127 (2010), which is hereby incorporated by reference in its entirety). After completing termination of a uORF, it was assumed that some translation factors remain associated with the ribosome, which facilitates the reinitiation process (Poyry et al., “What Determines Whether Mammalian Ribosomes Resume Scanning After Translation of a Short Upstream Open Reading Frame?” Genes Dev. 18(1):62-75 (2004), which is hereby incorporated by reference in its entirety). However, this mechanism is widely considered to be inefficient. From the GTI-seq data set, about half of the uORFs were separated from the main ORFs. Compared to transcripts with overlapping uORFs that must rely on leaky scanning to mediate the downstream translation, repressed aTIS initiation was observed in transcripts containing separated uORFs. It is likely that the ribosome reinitiation mechanism plays a more important role in selective translation under stress conditions (Vattem et al., “Reinitiation Involving Upstream ORFs Regulates ATF4 mRNA Translation in Mammalian Cells,” Proc. Natl. Acad. Sci. U.S.A. 101(31):11269-11274 (2004), which is hereby incorporated by reference in its entirety).

Biological Impacts of Alternative Translation Initiation

One consequence of alternative translation initiation is an expanded proteome diversity that has not been and could not be predicted by in silico analysis of AUG-mediated main ORFs. Indeed, many eukaryotic proteins exhibit a feature of NH₂-terminal heterogeneity presumably due to alternative translation. Protein isoforms localized in different cellular compartments are typical examples, because most localization signals are within the NH₂-terminal segment (Chang et al., “Translation Initiation From a Naturally Occurring Non-AUG Codon in Saccharomyces Cerevisiae,” J. Biol. Chem. 279(14):13778-13785 (2004) and Porras et al., “One single In-Frame AUG Codon is Responsible for a Diversity of Subcellular Localizations of Glutaredoxin 2 in Saccharomyces cerevisiae,” J. Biol. Chem. 281(24):16551-16562 (2006), which are hereby incorporated by reference in their entirety). Alternative TIS selection could also produce functionally distinct protein iso forms. One well-established example is C/EBP, a family of transcription factors that regulate the expression of tissue-specific genes during differentiation (Descombes et al., “A Liver-Enriched Transcriptional Activator Protein, LAP, and a Transcriptional Inhibitory Protein, LIP, are Translated from the Same mRNA,” Cell 67(3):569-579 (1991), which is hereby incorporated by reference in its entirety).

When an alternative TIS codon is not in the same frame as the aTIS, it is conceivable that the same mRNA will generate unrelated proteins. This could be particularly important for the function of uORFs, which are often separated from the main ORF and encode short polypeptides. Some of these uORF peptide products directly control the ribosome behavior, thereby regulating the translation of the main ORF. For instance, the translation of S-adenosylmethionine decarboxylase is subject to the regulation by the six amino acid product of its uORF (Hill et al., “Cell-Specific Translational Regulation of S-adenosylmethionine Decarboxylase mRNA. Dependence on Translation and Coding Capacity of the Cis-Acting Upstream Open Reading Frame,” J. Biol. Chem. 268(1):726-731 (1993), which is hereby incorporated by reference in its entirety). The alternative translational products could also function as biologically active peptides. A striking example is the discovery of short ORFs (“sORF”s) in noncoding RNAs of Drosophila that produce functional small peptides during development (Kondo et al., “Small Peptides Switch the Transcriptional Activity of Shavenbaby During Drosophila Embryogenesis,” Science 329(5989):336-339 (2010), which is hereby incorporated by reference in its entirety). However, both computational prediction and experimental validation of peptide-encoding short ORFs within the genome are challenging. The present invention represents a potential new addition to the expanding ORF catalog by including novel ORFs from ncRNAs.

The enormous biological breadth of translational regulation has led to an enhanced appreciation of its complexities. Yet, the current endeavors aiming to understand protein translation have been hindered by technological limitations. Comprehensive cataloging of global translation initiation sites and the associated ORFs is just the beginning in unveiling the role of translational control in gene expression. A systematic, high-throughput method like GTI-seq offers a top-down approach, in which one can identify a set of candidate genes to study intensively. GTI-seq is readily applicable to broad fields of fundamental biology. For instance, applications of GTI-seq in different tissues will facilitate the elucidation of the tissue-specific translational control. The illustration of altered TIS selection under different growth conditions will set the stage for future investigation of translational reprogramming during organismal development as well as in human diseases.

What is claimed:

1. A method for identifying a translation initiation site on an mRNA, said method comprising:
providing a first mRNA in an environment suitable for translation;
contacting the first mRNA with a first translation inhibitor to preferentially stabilize one or more initiation ribosomes at translation initiation sites on the first mRNA;
providing a second mRNA in an environment suitable for translation, wherein the second mRNA has a nucleotide sequence that is substantially similar to a nucleotide sequence of the first mRNA;
contacting the second mRNA with a second translation inhibitor different from the first translation inhibitor to stabilize one or more initiation ribosomes and one or more elongation ribosomes on the second mRNA; and
comparing the location of ribosomes stabilized on the first mRNA to the location of ribosomes stabilized on the second mRNA, wherein ribosomes stabilized at a location on the first mRNA at a higher density than ribosomes stabilized at the same location on the second mRNA identifies the location as a translation initiation site on the first and second mRNAs.

2. The method according to claim 1, wherein the first translation inhibitor binds to the ribosome after the ribosome is assembled at the translation initiation site.

3. The method according to claim 2, wherein said binding permits the formation of a first peptide bond in translation of the mRNA.

4. The method according to claim 3, wherein said first translation inhibitor is lactimidomycin.

5. The method according to claim 1, wherein the first translation inhibitor stabilizes ribosomes at translation initiation sites on the first mRNA and not at elongation sites on the first mRNA.

6. The method according to claim 5, wherein the second translation inhibitor is cycloheximide.

7. The method according to claim 1, wherein the first translation inhibitor blocks translocation of initiation ribosomes from the translation initiation site.

8. The method according to claim 1, wherein the translation initiation site is an AUG codon.

9. The method according to claim 1, wherein the translation initiation site is a codon other than AUG.

10. The method according to claim 1, wherein the nucleotide sequence of the second mRNA that is substantially similar to a nucleotide sequence of the first mRNA comprises a nucleotide sequence of at least 25 residues.

11. The method according to claim 1, wherein the nucleotide sequence of the second mRNA that is substantially similar to a nucleotide sequence of the first mRNA comprises a nucleotide sequence of at least 50 residues.

12. The method according to claim 1 further comprising:
contacting one or both of the first and second mRNAs with a compound capable of causing dissociation of elongating ribosomes from the first and/or second mRNA.

13. The method according to claim 12, wherein the compound is puromycin.

14. A kit for identifying a translation initiation site on an mRNA, said kit comprising:
a first translation inhibitor capable of preferentially stabilizing initiation ribosomes at translation initiation sites on an mRNA;
a second translation inhibitor different from the first translation inhibitor, wherein the second translation inhibitor is capable of stabilizing initiation ribosomes and elongation ribosomes on an mRNA; and
instructions for (i) contacting a first mRNA with the first translation inhibitor and a second mRNA with the second translation inhibitor and (ii) comparing the location of ribosomes stabilized on the first mRNA to ribosomes stabilized on the second mRNA to identify translation initiation sites on the first and second mRNAs.

15. The kit according to claim 14, wherein the first translation inhibitor binds to a ribosome after the ribosome is assembled at the translation initiation site.

16. The kit according to claim 15, wherein said binding permits the formation of a first peptide bond in translation of the mRNA.

17. The kit according to claim 16, wherein said first translation inhibitor is lactimidomycin.

18. The kit according to claim 14, wherein the first translation inhibitor stabilizes ribosomes at translation initiation sites on the first mRNA and not at elongation sites on the first mRNA.

19. The kit according to claim 18, wherein the second translation inhibitor is cycloheximide.

20. The kit according to claim 14, wherein the first translation inhibitor blocks translocation of initiation ribosomes from the translation initiation site.

21. The kit according to claim 11, wherein the translation initiation site is an AUG codon.

22. The kit according to claim 11, wherein the translation initiation site is a codon other than AUG.

23. The kit according to claim 11 further comprising:
a compound capable of causing dissociation of elongating ribosomes from the first and/or second mRNA and instructions for contacting one or both of the first and second mRNA with the compound to cause dissociation of elongating ribosomes.

24. The kit according to claim 23, wherein the compound is puromycin.

25. The kit according to claim 14, wherein the second mRNA has a nucleotide sequence that is substantially similar to a nucleotide sequence of the first mRNA.

26. The kit according to claim 25, wherein the nucleotide sequence of the second mRNA that is substantially similar to a nucleotide sequence of the first mRNA comprises a nucleotide sequence of at least 25 residues.

27. The kit according to claim 25, wherein the nucleotide sequence of the second mRNA that is substantially similar to a nucleotide sequence of the first mRNA comprises a nucleotide sequence of at least 50 residues.
